(19) United States
(12) Patent Application Publication
(10) Pub. No.: US 2003/0003533 A1
(43) Pub. Date: Jan. 2, 2003

(54) Method for isolation in vitro differentiated somatic cells

(76) Inventor: Wolfgang M. Franz, Wessling (DE)

Correspondence Address: BIRCH STEWART KOLASCH & BIRCH, PO BOX 747, FALLS CHURCH, VA, 22040-0747, US

(21) Appl. No.: 10/049,825

(22) PCT Filed: Mar. 26, 2001
(86) PCT No.: PCT/EP01/03412

(30) Foreign Application Priority Data
Mar. 24, 2000 (DE) 100 14 690.2

(51) Int. Cl. C12P 21/02; C12N 5/06; C07H 21/04; C07K 14/435

(52) U.S. Cl. 435/69.1; 435/320.1; 435/325; 530/350; 536/23.5

(57) ABSTRACT

The invention relates to an expression cassette that comprises, under the genetic control of an organ- or tissue-specific promoter and optionally one or more other regulatory elements 3′ downstream of the promoter, the coding nucleotide sequence of at least one non-immunogen receptor localized on the cell surface. The invention further relates to the vectors produced therefrom, to the host cells containing said vectors, to a method for purifying differentiated somatic cells using said constructs and to the use of said somatic cells in therapy.

This invention relates to novel processes for producing and selectively isolating differentiated, transgenic, somatic body cells, for example ventricular cardiomyocytes, the genetic constructs necessary for this purpose and the vectors produced with them, the host cells containing these vectors and the therapeutic use of transgenic differentiated somatic cells.

In the most varied fields of medicine, for example in neurology, dermatology, osteology, or cardiology, there is a need for allogenic or syngenic replacement tissues or replacement cell material. Therefore there is great interest in obtaining differentiated somatic body cells from pluripotent progenitor cells, for example embryonic stem cells. Embryonic stem cells (ES cells) have been known in science for a long time. They have been characterized on the mouse model. They have the extraordinary ability to differentiate essentially into each of the 210 different cell types of the human body.

Thomson et al. (1998), Science 282, 1145-1147 has recently been successful in isolating human ES cells from human blastocysts. In their physiological properties they are identical to those of mice. With isolation of human ES cells, successful cell-mediated gene therapy on humans is within reach. The importance of this development will be briefly illustrated using the example of cardiology.

Cardiac insufficiency describes the inability of the heart to transport blood and oxygen to the organs in an amount which meets the needs of the body. In 1996 in Germany more people died of chronic cardiac insufficiency than acute cardiac infarction. The main cause of this is that too few donor hearts for life saving-hearts transplants are available and the waiting time at present is roughly nine to twelve months.

Currently available alternative methods to heart transplantation are only of limited success or have not developed far enough. In general clinical practice in the first step mainly drug therapy of chronic cardiac insufficiency is used for myocardial relief and contraction support. This is followed by surgical methods, such a myocardial resection after Batista, according to which damaged tissue is mechanically removed, cardiomyoplasty, i.e. support of cardiac function by autologous skeletal musculature and implantation of an artificial heart. In the experimental phase there is still the controversial xenotransplantation, therefore the implantation of a donor heart from a pig or monkey into a human. The latter applies not only to ethical objections, but also to medical risks associated with xenotransplantation. On the one hand the jump of alien disease pathogens to humans cannot be precluded. On the other hand, xenotransplantation always presupposes the use of immune-suppressing drugs which do protect the transplanted heart from an immune reaction, but make the entire organism vulnerable to daily germs and cancer formation.

The probably most promising alternative to heart transplants would be autologous cell transplantation. In cardiology this is defined as injection of healthy cells directly into the damaged area of the heart.

Cardiomyocytes in contrast to transversely striated skeletal muscle shortly before birth lose the ability to divide so that in case of injury or damage to the myocardium irreversible cell losses and function losses occur. In the case of a cardiac infarction this means replacement of cardiac musculature by connective tissue with the formation of a scar. This could be counteracted by the transplantation of replacement muscle cells.

The technical feasibility of cellular transplantation in test animals such as mice, rats, and dogs, has been demonstrated not only by transplantation of fetal, neonatal, and adult cardiomyocytes, but also by the injection of cells foreign to the organ, like myoblasts, skeletal muscle satellite cells and cardiac cells (Klug et al., (1996) J. Clin. Invest. 98, 216); Koh et al., (1995) J. Clin. Invest. 96, 2034; Schwarz et al., (1998) Z. Kardiol. 87, 1). Histological-morphological studies confirm that both myoblasts and also cardiomyocytes form myocardial transplants with intercellular connections, such as gap junctions and desmosomes, and survive 8 to 10 weeks (Koh et al, loc. cit.; Soonpaa et al., (1994) Science 264, 98). Koh et al. were able to show that genetically modified transplanted myoblasts in the recipient myocardium express recombinant growth factors over the long term and thus in the boundary area between the transplant and recipient myocardium can induce endothelial DNA synthesis with subsequent angiogenesis (Koh et al., (1995) J. Clin. Invest. 95, 114). It is partially unexplained whether the cell transplants in fact have positive effects on the myocardium or whether over the longer term they do not cause for example electrical and structural instabilities. The problem in the acquisition of cardiomyocytes is the manipulation of cells. It is indeed possible to force cardiomyocytes back into the cell cycle, for example by inserting tumor genes, but at the cost of physiological cell properties. The electrophysiological, pharmacological and immunohistological characteristics which are typical of ventricular cardiomyocytes are thus lost.

The basic prerequisite for success of a cell transplantation approach is the presence of a homogeneous cell population. ES cells in addition to their ability to differentiate into any cell type also have one special disadvantage. If they are injected in the undifferentiated state into mice, tumors, so-called teratocarcinomas, form. Before ES cells can be therapeutically used, it must therefore be ensured that all cells are fully differentiated such that there is no danger of uninhibited reproduction of the cells or formation of unwanted tissue types. Stringent purification of the desired cells type, in the case of cardiology, of cardiac muscle cells, is therefore an absolute necessity for the safety of the recipient.

Loren J. Field (J. Clin. Invest. (1996, 98, 216) was able to concentrate spontaneously differentiated cardiomyocytes with a degree of purity exceeding 99%. To reach this objective, they first introduced an antibiotic-resistance gene into the murine ES cells. It was designed such that it is expressed solely in cardiomyocytes. After the ES cells were given time to differentiate and after all cells without the resistance gene were killed by adding enough antibiotic, it was possible to obtain an essentially pure population of cardiomyocytes. After transplantation of these cells into mouse myocardia, they could be detected up to an interval of more than 7 weeks. The main defects of this method are mainly the toxicity of the G418-containing medium used, the low yield of the cells obtained in this way and the fact that the described cells were not human, but murine cells.

Although cardiomyocyte transplantation on the animal model has been repeatedly done successfully, promising performance on humans for the aforementioned reasons and mainly due to the unavailability of human ventricular cardiomyocytes is not possible. Similar problems can be expected in the acquisition and transplantation of other somatic cells.

### BRIEF DESCRIPTION OF THE INVENTION

Therefore the object of this invention is to make available improved means and processes for acquisition of differentiated somatic cells. In particular, the object was to make available improved means and processes for acquisition of differentiated ventricular cardiomyocytes.

The aforementioned object was surprisingly achieved by making available a novel expression cassette for the genetic modification of the somatic cells to be isolated or pluripotent precursor cells of somatic cells. This expression cassette which is suitable for administration to the pluripotent precursor cells of a mammal, for example as a component of an expression vector, is characterized in that under the genetic control of an organ-specific or tissue-specific promotor and optionally one or more other regulatory elements 3′-downstream of the promotor, functionally or operatively linked, it encompasses the coding nucleotide sequence of at least one receptor which is located on the cell surface and which is expressed after induction of cell differentiation. Here the expressed receptor for a mammal to which the differentiated cells are to be administered is not immunogenic.

By the combination of-an organ-specific or tissue-specific promotor and a receptor located on the cell surface, with organ-specific or tissue-specific expression controlled by the aforementioned promotor in the desired subpopulation of differentiated somatic cells, rapid, careful and selective isolation of a specific cell population using the bonding affinity of the receptor to a correspondingly bonding partner or ligand is enabled.

### DETAILED DESCRIPTION OF PREFERRED EMBODIMENTS OF THE INVENTION

Depending on the desired differentiated cell population which is to be isolated, one skilled in the art can select the cell-specific or organ-specific promotor which is most suitable at the time and a suitable receptor.

Nonlimiting examples for suitable promoters are the following:

| Promotor | Specificity | Location |
|---|---|---|
| MLC-2 | cardiac muscle | PCT/DE 96/02181 |
| SMHC | smooth vascular muscle | DE-A-196 20 308 |
| Insulin promotor | beta cells | |
| AFP promotor | liver | |

Reference is made expressly to the disclosure of the aforementioned locations.

For the choice of the receptor or its extracellular area it is decisive that after expression on the cell surface it has a binding affinity to a ligand and low immunogenicity in a later recipient organism. Preferably the receptor or its extracellular area should be “non-immunogenic” in the recipient organism.

A non-immunogenic” receptor which can be used as claimed in the invention or its extracellular area should have at least one of the following properties:

a) it is recognized in expressed form on the differentiated somatic cell of the immune system of a mammal to which the somatic cells differentiated as claimed in the invention are to be administered, not as “alien”, but as “autologous”;

b) it is not expressed under native conditions, i.e. under normal development conditions on the undifferentiated pluripotent precurso cells of the mammal, especially that mammal to which later the cells differentiated as claimed in the invention are to be administered;

c) it is essentially not expressed by the organ-specific or tissue-specific cells of a mammal which are to be genetically modified using the expression cassette, under native conditions or is expressed only in a later development stage of the cells.

The receptor molecule can be expressed either in its original native form or in a genetically altered form as a “functional equivalent”. A “functional equivalent” of the receptor furthermore has qualitatively comparable bonding properties with the native molecule, but however for example can have changes in the amino acid sequence (additions, substitutions, insertions, deletions) by which for example the bonding affinity of the receptor to the corresponding ligand is increased or decreased. Especially changes by which the intracellular domain of the native receptor is deleted in whole or in part are also conceivable.

Especially useful receptors are those which are natively expressed by the cell system of the autologous immune system, for example by B or T cells. Examples can include the following: CD2 to CD10, CD11a, CD11b, CD14, CD15s, CD16, CD17, CD18 to CD21, CD25, CD27 to CD32, CD38 to CD40, CD41A, CD41b, CD44, CD45, CD45RA, CD47, CD49b, d, e and f, CD56, CD58, CD59, CD61, CD63, CD64, CD69, CD74, CD78, CD79b, CD80, CD81, CD83, CD86, CD87, CD89, CD90, CD92, CD93, CD95, CD97, CD98, CD100, CD101, CD122, CD128, CD130, CD132, CD134, CD137, CD152, CD154, CD158a, CD161 to CD163, CD165, ICAM-1, LFA-1, LFA-3, CTLA-4, B7, hB7-PR1, BSL2vc, BSL3, ICOS, PD-1, HLA antigens and the like.

The desmoglein receptor can be named as another suitable molecule.

The CD4 receptor is preferred.

Suitable ligands are the natural bonding partners of the aforementioned receptor molecules or immunoglobulins, such as especially monoclonal antibodies or fragments thereof.

| Receptor | Ligand |
|---|---|
| CD4 | anti-CD4 antibody |

One preferred embodiment of the expression cassette as claimed in the invention is characterized in that the coding nucleotide sequence is contained in a polycistronic, preferably bi-cistronic gene which moreover comprises the coding sequence for at least one other gene product, selected from among a marker gene and a first therapeutic gene. Preferably the bi-cistronic gene comprises the regulator sequence of an internal ribosome entry side (IRE-S) (for example, commercially available from Clontech). One such polycistronic construct offers the additional unexpected advantage that under the genetic control of the upstream promotor simultaneous expression of the receptor and the other gene takes place. If the other gene is a marker gene, such as for example EGFP (green fluorescing protein), this facilitates the characterization of the cells isolated using the receptor. If the other gene is a therapeutic gene, its tissue-specific or organ-specific expression is guaranteed and thus the risk of unwanted side effects by uncontrolled expression in other tissues or organs is essentially precluded.

In addition to the promotor, if desired, other regulatory elements can be contained in the expression cassette, such as amplification signals, enhancers, polyadenylation sequences, replication sources, reporter genes, marker genes and the like.

In the constructs as claimed in the invention a surface antigen which has immunological affinity for the immunoglobulin molecule is used as the receptor molecule. The immunoglobulin molecule is preferably a monoclonal anti-receptor antibody or a receptor-binding fragment such as for example a FAB or F(ab′)2 or Fv fragment thereof.

For easier isolation of the receptor-expressing cells the ligand, for example the antibody, can be present in immobilized form, for example bound to a fixed carrier or linked to paramagnetic microspheres, so-called microbeads.

Preferably as claimed in the invention the CD4 antigen or shortened fragment thereof, preferably a molecule which is shortened by the intracellular domain and which thus furthermore comprises the transmembrane and extracellular domain, is used as the surface antigen.

According to another embodiment the expression cassette can contain a selectable marker, for example, a resistance gene. Basically any known resistance genes can be used. Examples are especially antibiotic resistance genes such as neomycin- and hygromycin-resistance gene.

Preferably the resistance gene and its assigned promotor are present in a reversible integrated form and can thus be removed again at a suitable instant, in any case before therapeutic use of the cell. The reversible incorporation of the resistance gene and its promotor is achieved for example by flanking it by LOX sequences (LoxP-promotor resistance gene-LoxP). Thus the foreign gene can be specifically removed by transient transfection of resistance gene-containing cells and expression of PKG-Cre (for example, described in Cellemand et al., (1998) Transg. Res. 7, 105).

An example of a group of first therapeutic genes which can be used as claimed in the invention are genes for angiogenesis factors. Preferred representatives of this group are the vascular endothelial growth factor (VEGF) gene, the basic fibroblast growth factor (bFGF) gene, the acidic fibroblast growth factor (aFGF) gene, the angiopoietin, activin and follicostatin gene. They are preferably a component of the aforementioned bi-cistronic gene.

In another preferred embodiment the construct encompasses moreover a second therapeutic gene, especially an immune suppression gene.

Preferably this gene is contained in independent form, i.e. with its own promotor. These constructs offer the advantage that a local immunosuppressive action can be mediated by gene therapy. The immunosuppressive gene products can be present membrane-based or preferably in secreted form. A suitable secreted immunosuppressing gene product is the CTLA4-Ig fusion protein.

Based on the preferred therapeutic use of the cell isolates as claimed in the invention in humans, especially those constructs are advantageous which have coding sequences which code essentially for human or humanized gene products. “Humanized” gene products are those in which to reduce their immunogenicity, non-human partial sequence areas are replaced by the corresponding human-typical sequence areas.

One especially preferred group of expression cassettes as claimed in the invention is characterized in that the ventricle-specific myosin-light chain-2 (MLC-2v) promotor is used as the organ-specific or tissue specific promotor. This promotor and suitable versions thereof are described in PCT/DE 96/02181, to which reference is expressly made. Examples for special forms of preferred expression cassettes are constructs which in the 5′-3′ direction comprise at least one of the following partial sequence successions:

a) MLC-2v promotor, CD4 extracellular and transmembrane domains, IRES, angiogenesis factor;

b) CMV enhancer, MLC-2v promotor, CD4 extracellular and transmembrane domains, IRES, angiogenesis factor;

c) CMV enhancer, MLC-2v promotor, CD4 extracellular and transmembrane domains, IRES, angiogenesis factor, PGK promotor, CTLA4-Ig fusion protein; and

d) CMV enhancer, MLC-2v promotor, CD4-extracellular and transmembrane domains, IRES, angiogenesis factor, LoxP, PGK promotor, resistance gene, LoxP, PGK promotor, CTLA4-Ig fusion protein;

here the CMV enhancer is a regulatory element from the cytomegalovirus and the PGK promotor is the promotor sequence for the enzyme phosphoglycerinkinase. These preferred constructs also advantageously comprise coding sequences which code essentially for human or humanized gene products.

A further subject matter of the invention relates to vectors such as plasmids or viral constructs, phages, phasmids, phagemids, transposons, cosmids or liposomes, comprising at least one of the above described expression cassettes. Preferably viral, especially adenoviral, constructs or liposome preparations are used. Preferred MLC-2-containing vectors are described in the aforementioned PCT/DE 96/02181.

Furthermore, the subject matter of the invention is a process for isolation of in vitro differentiated organ-specific or tissue-specific body cells of a mammal,

a) an organ-specific or tissue-specific expression vector according to the aforementioned definition being introduced into pluripotent precursor cells, especially chosen from among embryonal stem cells, primordial cells and bone marrow stroma cells of a mammal;

b) transgene-positive cells being selected;

c) optionally present resistance genes being removed from the selected cells;

d) in the cells obtained in this way, differentiation into a cell population comprising the desired organ-specific or tissue-specific somatic cells being induced and if necessary a single cell preparation being produced; and

e) the receptor-expressing differentiated somatic cells being affinity-purified using receptor-specific ligands.

The subject matter is especially also a process for producing ventricular cardiomyocytes,

a) the above described ventricle-specific expression vector being introduced into pluripotent precursor cells, especially chosen from among embryonal stem cells, primordial cells and bone marrow stroma cells of a mammal;

b) transgene-positive cells being selected;

c) optionally present reversibly integrated resistance genes being removed from the selected cells;

d) in the cells obtained in this way, differentiation into a cell population comprising the cardiomyocytes being induced and if necessary a single cell preparation being produced; and

e) the receptor-expressing differentiated ventricular cardiomyocytes being affinity-purified using receptor-specific ligands.

One version of the aforementioned process is characterized in that LoxP-flanked resistance genes are used as reversibly integrated resistance genes, and to remove this expression vector which codes the cells with Cre-recombinase transient transfection is done.

In another version of the aforementioned process, embryonal stem cells are used which have been obtained conventionally from

a) blastocysts or

b) enucleated oocytes into which the nucleus of a differentiated adult somatic body cell has been transferred.

In the process as claimed in the invention, the pluripotent precursor cells are genetically manipulated with an expression construct as claimed in the invention. For example, a cell population using electroporation can be transfected in the conventional manner. It is especially well suited in cells which can be clonally isolated. If clonal isolation is difficult, then using viral vectors, for example adenoviral constructs, high gene transfer rates into the pluripotent cells can be achieved. After induction of differentiation, under the control of the organ-specific or tissue-specific promotor, the receptor is expressed specifically in the desired subpopulation of the differentiated cells. Using the specific interaction between the receptor on the cell surface and an assigned ligand, isolation of the desired differentiated cells can take place.

Preferably, in the process as claimed in the invention the receptor-specific ligands are coupled to paramagnetic microbeads so that the ligand-marked cells are separated from the unmarked cells in a magnetic field.

The process as claimed in the invention can be carried out essentially with pluripotent stem cells of any mammals, for example humans, mice, rats, pigs, cattle, dogs, rabbits, hamsters.

The aforementioned processes are used especially to produce autologous human somatic cells, the pluripotent predecessor cells being obtained from an autologous human donor.

The subject matter of the invention is especially transgenic somatic body cells which can be obtained using a process as claimed in the invention. They are used especially for preferably autologous cell transplantation or for gene therapy, especially for cell-mediated gene transplantation in the most varied disease states. Nonlimiting examples for possible indications are ischemic and dilatative cardiomyopathy.

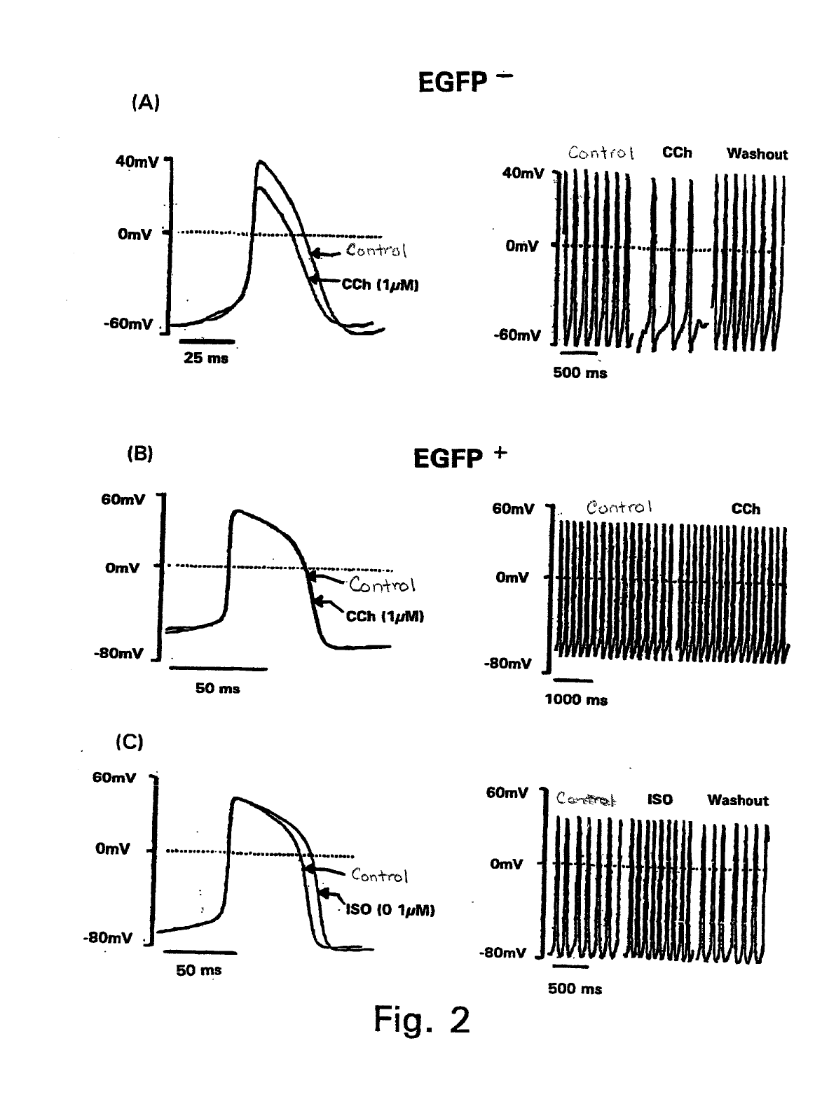

The subject matter is especially cardiomyocytes with an electrophysiologically ventricular property spectrum, i.e. a membrane potential of roughly −70 mV which is typical of ventricular cardiomyocytes, a potential length of roughly 118 ms and an overshoot of roughly 34 mV, carbachol as the agonist of the muscarinic receptor having no effect on the membrane potential and the length of the action potential. Moreover, continuation of the action potential after treatment of cells with the beta-adrenergic agonist isoprenaline is typical for ventricular cardiomyocytes (compare also FIG. 2).

Production and acquisition of cardiac muscle cells from various pluripotent progenitor cells is detailed in the following segments.

a) Production from ES Cells:

Current knowledge about the acquisition and handling of EC cells originates for the most part from studies of murine ES cells. They were obtained for the first time in 1981 from a mouse embryo in the 100 cell stage. The embryo at this development times is called a blastocyst. It measures a few millimeters and consists of a hollow sphere which is thickened at one point to the inside toward the inner cell mass. Under natural conditions the fetus develops from it in the uterus. If the blastocyst is grown in any case in a Petri dish, the outer membrane collapses and the inner cell mass begins to divide. In this stage the cells can kept for an unlimited time interval. Their pluripotency, i.e. their ability to differentiate into almost any cell type, continues to be preserved.

ES cells preserve their undifferentiated state as long as they find the cytokine LIF (leukemia inhibitory factor) secreted by the so-called feeder cells in their nutrient medium. If the feeder cells or LIF are removed and the ES cells are prevented from attaching to the substrate, for example by culture of the ES cells on bacteria plates or in so-called “hanging drops”, they begin to differentiate. During differentiation so-called “embryoid bodies” form. The direction of differentiation initially remains unpredictable, but the repertory of cells differentiating in vitro is much smaller than after injection into a blastocyst; presumably caused by the cell environment which is chemically defined differently. If the ES cells are grown in the presence of stroma cells, intensified formation of hematopoietic cells occurs. The same applies when methyl cellulose is added to the culture medium. The addition of retinic acid (RA), depending on the concentration and instant of stimulation, leads to accumulation of cardiac or neural cells. The corresponding methods are described in DE 44 41 327 or WO 96/16163 for example.

Aside from minor deviations in the working protocol, the aforementioned handling and differentiation procedure described for murine ES cells can also be applied to human ES cells. While in murine blastocysts, after the start of in vitro differentiation, the outer cell layer (trophoblast) is broken down relatively quickly, this is not the case in human blastocysts. In order to protect the inner cell mass from death, therefore the trophoblast must be mechanically removed in the in vitro differentiation of human blastocysts (described by Thomson, loc. cit.)

b) Alternative Production Possibilities:

In vitro differentiation of ES cells is not the only way to obtain human cardiomyocytes. Cardiomyocytes are likewise accessible by in vitro differentiation of primordial cells, precursor cells of egg and sperm cells which are obtained from human fetal ovaries or testes. Establishment of human pluripotent primordial cells is described for example in Shamblott et al., (1998) Proc.Natl.Acad.Sci., 95, 13726.

Moreover, cardiomyocytes are accessible from in vitro differentiated oocytes which have been manipulated by nucleus transfer techniques. Suitable methods are described for example by Wakayama, T. et al., (1998), Nature, 394, 369; and Wilmut, I. et al., (1997), Nature, 385, 810. This approach enables especially autologous cell transplantation, i.e. the possibility of obtaining and transplanting autologous cardiomyocytes.

Furthermore, cardiomyocytes are accessible by differentiation of bone marrow stroma cells. A process for producing murine cardiomyocytes is described by Makino et al., (19-99) J. Clin. Invest. 103, 697.

One important point which has not yet been examined in detail is the fact that each of the aforementioned in vitro differentiation methods leads to a mixture of different cell types. Thus, for example in in vitro differentiated murine ES cells the percentage of myocardial cells is only 5%. For in vitro differentiation of human primordial or ES cells similar figures must be expected. This means that only a fraction of the entire in vitro differentiated cell population ever corresponds to the desired cell type. This percentage can possibly be doubled by suitable growth conditions, such as the addition of chemical inductors, but at first promising cell transplantation cannot be undertaken with one such cell mixture. Conventional purification methods are very complex and time-consuming and cause the initially described undesirable changes of physiological cell properties. Only by using the approach as claimed in the invention for marking and purification of differentiated cardiomyocytes can this problem be satisfactorily circumvented.

The invention is detailed using the following examples and with reference to the attached figures.

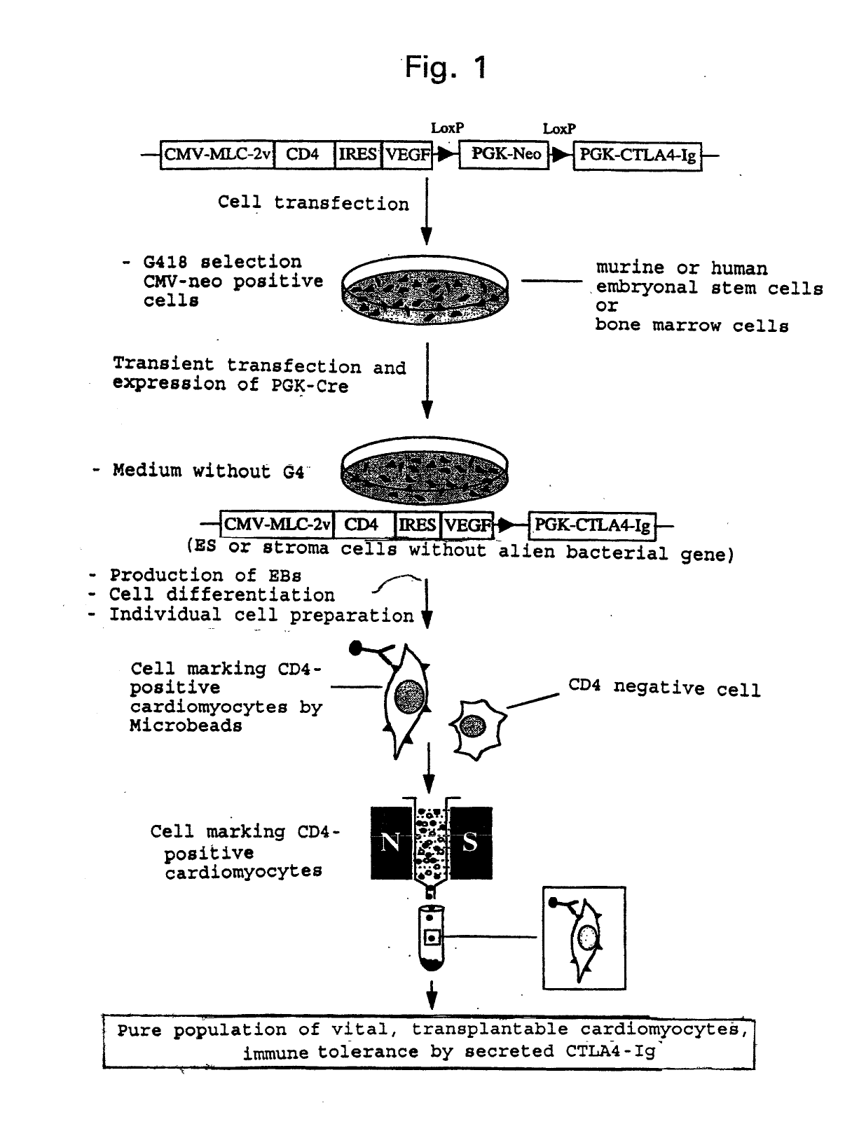

FIG. 1 shows marking and purification of ventricular cardiomyocytes. The expression plasmid used for transfection of pluripotent cells (murine or human ES and bone-marrow stroma cells or primordial cells) contains the following subunits: (1) a bi-cistronic gene consisting of a CMV-MLC2v promotor which is specific to ventricular cardiomyocytes and which regulates a shortened CD4 surface protein and the therapeutically effective VEGF gene; (2) an independent PGK neo-gene which is flanked by LoxP sequences and which is used for selection of the positively transformed cells and is removed by transient expression of a Cre expression vector before transplantation; and (3) a PGK-CTLA4-Ig fusion protein gene which as an independent unit is intended to contribute to imparting to the cell transplant immunity to a rejection reaction proceeding from the recipient tissue.

After transfection of cells, for example, by means of electroporation, selection of positively recombined cells in the G418-containing medium occurs. G418-resistant cell clones are transiently transfected with a PGK-Cre expression plasmid, by which LoxP-flanked alien gene areas are removed from the genome of the cell, i.e. the gene construct infiltrated into the cells at this time no longer contains genes foreign to the patient. After producing the embroid bodies (EBs) differentiation of the pluripotent cells follows, for example into cardiac muscle cells. The heterogenous cell mixture which forms is then subject to individual cell preparation. CD4-positive cells can thereupon be marked by means of anti-CD4 antibodies and purified via separation in a magnetic field. The cell population obtained in this way consist of ventricular cardiomyocytes which can be used directly for transplantation studies;

FIG. 2 shows the electrophysiological characterization of ventricular cardiomyocytes. (A) EGFP-negative cardiomyocytes show the typical action potential of early cells with a depolarized membrane potential of roughly −56 mV and an only briefly lasting action potential over roughly 86 ms. Moreover, they show a negative chronotropic effect relative to treatment with the muscarinic agonist carbachol (CCh). (B) EGFP-positive cardiomyocytes conversely show typical properties of the ventricular type: negative membrane potential of roughly −70 mV and a duration of the action potential of roughly 118 ms. They show a clearly detectable plateau phase which is caused by long-lasting calcium inflow through calcium channels of the L-type. CCh shows no effect on ventricular cardiomyocytes. Treatment with the beta-adrenergic agonist isoprenaline (Iso) however leads, as shown in (C), to a prolongation of the action potential, in turn typical for cardiomyocytes of the ventricular type.

To the extent there is no separate information, the experiments are carried out using standard molecular biological and cell biological methods (compare, for example, Shambrook et al., Molecular Cloning, A Laboratory Manual, 2nd ed. (1989) Cold Spring Harbor Laboratory.

### EXAMPLE 1

Production of an expression vector comprising the following partial sequence succession:

- // CMV-MLC-2v/CD4/IRES/EGFP//PGK-Meo//PGK-CTLA4-Ig// -
Gene 1 Gene 2 Gene 3

1. First, a CMV-MLC-2v-EGFP plasmid is produced. The CMV enhancer element consisting of 590 base pairs (Boshart et al. (1985) Cell 41, 521) was produced by means of PCR from the plasmid CMV-EGFP which is available from Clontech and then via the interfaces SacI, BamHI cloned into the expression vector pEGFP-1 (Clontech) (pCMV-EGFP). To be able to insert the MLC2v promotor it was first isolated as a 2.1 kb Kpn1-EcorR1 DNA fragment from the rat genome (Henderson et al., (1989) JBC. 2641 18142), inserted into the EcoRI, XhoI interfaces of the pEGFP-1 (MLC2v-EGFP), then cut out of it by means of XbaI, XhoI-digest and inserted into the BamHI, XhoI interfaces of pCMV-EGFP (CMV-MLC2v-EGFP).

A CD4 expression plasmid is available from Miltenyi Biotec (Germany). The coding areas of the CD4 molecule shortened by the intracellular part are ligated to the 3′-end of the CMV-MLC2v enhancer/promotor construct and the IRES-EGFP cassette following in the 3′ direction is recloned from an expression vector ordered from Clontech. The bi-cistronic gene obtained in this way is intended to guarantee simultaneous ventricle-specific expression of the marker genes CD4 and EGFP. CD4 is used later for actual purification of the cardiomyocytes. EGFP is used to characterized the cells obtained by the purification. The above described vector moreover contains an independent PGK-neo gene and a CTLA4-Ig fusion gene described by the working group of Gainer et al., (1997) Transl. 63, 1071.

In addition, an identical vector without the PGK-CTLA4-Ig cassette is used as the control vector to later determine in mice or rats the biological effect of CTLA4-Ig.

2. The vector which is illustrated above and which consists of 3 genes is linearized and transfected by electroporation into murine ES cells and bone marrow stroma cells of the human or rat. Bone marrow stroma cells are isolated in the conventional manner from the femurs of Wistar rats and purified via a Perkoll gradient. Human mononuclear bone marrow cells purified via a Perkoll gradient can also be ordered from CellSystems.

The cells are kept in gelatinized Petri dishes, the ES cells being cultivated at 37° C., the bone marrow stroma cells at 33° C. Transformed cells can be selected via a neomycin resistance cassette. In geneticin (G418)-resistant ES cell clones, thereupon according to information in WO 96/16163 differentiation is induced by removing LIF. Roughly 25 days after the start of differentiation the CD4-expressing cardiomyocytes from the formed embryoid bodies are purified.

Analogously to the protocol optimized by Makino et al., loc. cit., for murine stroma cells, in the stroma cells of rats and man differentiation is also stimulated by administering 5′-azacytidin.

3. For purification of CD4-expressing cardiomyocytes, first an individual cell preparation is produced from the embryoid bodies. The enzymes which are typically used for this purpose, collagenase and trypsin, cannot be used since they would break down the CD4 surface molecule used. A non-enzymatic cell dissociation solution which is available for example from Sigma affords relief. Individual cells are then incubated with anti-CD4 antibodies. The antibodies bind to their corresponding antigen and thus mark all CD4-expressing cells. Since the antibodies for their part are coupled to paramagnetic “microbeads”, it is possible to isolate the complex consisting of the CD4-expressing cardiomyocyte, anti-CD4 antibodies and the “microbeads” in the conventional manner via an external magnetic field.

The cells marked by paramagnetic “microbeads” are exposed to the field of a strong magnet in a column supplied by Miltenyi Biotec. The column material consists of ferromagnetic particles which are provided with a cell-friendly hydrophilic coating. The cells marked by the “microbeads” first remain on the separation column, while unmarked cells are washed out. Turning off the magnet ultimately leads to elution of the marked cells. Complete purification takes roughly 1-2 hours. Detachment of the paramagnetic beads from the cell surface it is not necessary due to their small size and their composition (iron oxide and polysaccharide). Separation is thus much faster and more protective of the cells than separation via a cell sorter.

4. The yield and composition of the purified cells is then determined via expression of the EGFP cassette by FACS analysis and fluorescence microscopy. The expression of CTLA4-Ig can be detected by means of monoclonal anti-CTLA4-Ig antibodies and indirect immunofluorescence likewise in FACS analysis and in ELISA.

### EXAMPLE 2

Production of an expression vector comprising the following partial sequence succession:

-//CMV-MLC-2v/CD4/IRES/VEGF//PGK-Meo//PGK-CTLA4-Ig//-

Analogously as described in example 1, a construct is described which comprises instead of the EGFP cassette the therapeutic VEGF-gene (Carmeliet, P., et al. (1999) Nature Med. 5, 495). The cardiomyocytes genetically altered in this way then act as “carriers” in order to apply the angiogenesis factor locally in the myocardium.

In general it applies that in the therapeutic use of expression constructs as claimed in the invention, such as for example the above described VEGF-expressing plasmids, all genes which are present in the transgenic expression vector should be of human origin to minimize the immune reaction of the body. This in turn means that in addition to the already replaced EGFP gene the still present bacterial neo-resistance gene must also be removed. The strategy used for removing alien genes is illustrated in FIG. 1. In the expression vector shown which can be used for therapeutic approaches the neo-cassette is flanked by non-copying, so-called LoxP signal sequences. If transgene-positive ES or stroma cells are transiently transfected with a Cre-expression vector (PGK-Cre) even before differentition into cardiomyocytes, the activity of the Cre-recombinase removes the LoxP-flanked gene areas from the genome, one of the LoxP sequences remaining in the genome (Selbert, S. (1999) BIUZ, 29, 70).

Proceeding from the aforementioned specific disclosure, one skilled in the art can undertake modifications of the specifically described methods using his technical knowledge to adapt them optionally to the special requirements of pluripotent precursor cells of varied origin.

1. Expression cassette, characterized in that under the genetic control of an organ-specific or tissue-specific promotor and optionally one or more other regulatory elements 3′-downstream of the promotor, it encompasses the coding nucleotide sequence of at least one non-immunogenic receptor which is located on the cell surface.

2. Expression cassette as claimed in claim 1, wherein the coding nucleotide sequence codes for a receptor which has at least one of the following properties:
a) it is not recognized in expressed form by the immune system of a mammal as “alien”;
b) it is not expressed under native conditions on the undifferentiated pluripotent precursor cells of the mammal;
c) it is essentially not expressed by the organ-specific or tissue-specific cells of a mammal which are to be genetically modified using the expression cassette, under native conditions.

3. Expression cassette as claimed in claim 1 or 2, wherein the coding nucleotide sequence of the receptor is contained in a poly-cistronic gene which moreover comprises the coding sequence for at least one marker gene and/or a first therapeutic gene.

4. Expression cassette as claimed in one of the preceding claims, wherein the receptor has affinity for a ligand.

5. Expression cassette as claimed in claim 4, wherein the receptor is a surface antigen which has immunological affinity for an optionally immobilized immunoglobulin molecule.

6. Expression cassette as claimed in claim 5, wherein the surface antigen is a CD4 antigen or shortened fragment thereof, preferably its extracellular and transmembrane domain.

7. Expression cassette as claimed in one of the preceding claims, wherein it moreover comprises a reversibly integrated resistance gene.

8. Expression cassette as claimed in claim 7, wherein the resistance gene is flanked by LoxP sequences.

9. Expression cassette as claimed in claim 2, wherein the marker gene is the EGFP gene.

10. Expression cassette as claimed in one of the preceding claims, wherein it moreover comprises a second therapeutic gene.

11. Expression cassette as claimed in claim 2 or 10, wherein the first and the second therapeutic gene are chosen independently of one another from among genes for angiogenesis factors, such as especially the vascular endothelial growth factor (VEGF) gene, the basic fibroblast growth factor (bFGF) gene, the acidic fibroblast growth factor (aFGF) gene, the angiopoietin, activin and follicostatin gene, and immune suppression genes, such as especially the CTLA4-Ig fusion gene.

12. Expression cassette as claimed in one of the preceding claims, with coding sequences which code essentially for human or humaized gene products.

13. Expression cassette as claimed in one of the preceding claims, wherein the ventricle-specific myosin-light chain-2 (MLC-2v) promotor is used as the organ-specific or tissue specific promotor.

14. Expression cassette as claimed in claim 13, wherein it comprises in the 5′-3′ direction at least one of the following partial sequence successions:
a) MLC-2v promotor, CD4 extracellular and transmembrane domains, IRES, angiogenesis factor;
b) CMV enhancer, MLC-2v promotor, CD4-extracellular and transmembrane domains, IRES, angiogenesis factor;
c) CMV enhancer, MLC-2v promotor, CD4 extracellular and transmembrane domains, IRES, angiogenesis factor, PGK promotor, CTLA4-Ig fusion protein; and
d) CMV enhancer, MLC-2v promotor, CD4-extracellular and transmembrane domains, IRES, angiogenesis factor, LoxP, PGK promotor, resistance gene, LoxP, PGK promotor, CTLA4-Ig fusion protein.

15. Expression cassette as claimed in one of claims 13 to 14, with coding sequences which code essentially for human or humanized gene products.

16. Vector comprising an expression cassette as claimed in one of claims 1 to 12.

17. Vector comprising an expression cassette as claimed in one of claims 13 to 15.

18. Process for isolation of in vitro differentiated organ-specific or tissue-specific somatic cells of a mammal,
a) an organ-specific or tissue-specific expression vector as claimed in claim 16 being introduced into pluripotent precursor cells, especially chosen from among embryonal stem cells, primordial cells and bone marrow stroma cells of a mammal;
b) transgene-positive cells being selected;
c) optionally present resistance genes being removed from the selected cells;
d) in the cells obtained in this way, differentiation into a cell population comprising the desired organ-specific or tissue-specific somatic cells being induced and if necessary a single cell preparation being produced; and
e) the receptor-expressing differentiated somatic cells being affinity-purified using receptor-specific ligands.

19. Process for producing ventricular cardiomyocytes,
a) the ventricle-specific expression vector as claimed in claim 17 being introduced into pluripotent precursor cells, especially chosen from among embryonal stem cells, primordial cells and bone marrow stroma cells of a mammal;
b) transgene-positive cells being selected;
c) optionally present reversibly integrated resistance genes being removed from the selected cells;
d) in the cells obtained in this way, differentiation into a cell population comprising the cardiomyocytes being induced and if necessary a single cell preparation being produced; and
e) the receptor-expressing differentiated ventricular cardiomyocytes being affinity-purified using receptor-specific ligands.

20. Process as claimed in claim 18 or 19, wherein LoxP-flanked resistance genes are used as reversibly integrated resistance genes and are transiently transfected to remove this expression vector which codes the cells with Cre recombinase.

21. Process as claimed in one of claims 18 to 20, wherein the embryonal stem cells are obtained from
a) blastocysts or
b) enucleated oocytes into which the nucleus of an differentiated adult somatic cell has been transferred.

22. Process as claimed in one of claims 18 to 21, wherein the receptor-specific ligands are coupled to paramagnetic microbeads and the ligand-marked cells are separated from the unmarked cells in a magnetic field.

23. Process as claimed in one of claims 18 to 22 for producing autologous human somatic cells, the pluripotent precursor cells being obtained from an autologous human donor.

24. Transgenic cardiomyocytes with an electrophysiologically ventricular property spectrum.

25. Transgenic cardiomyocytes as claimed in claim 24, with one, several or all of the following electrophysiological features:
a) membrane potential in the range of roughly −68.6±2.8 MV;
b) membrane potential length in the range of roughly 118.3±15.2 mV;
c) overshoot in the range of roughly 34±3.9 mV;
d) no response of the membrane potential and action potential length to 1 μm carbachol; and
e) prolongation of the action potential by administering 0.1 μM isoprenaline.

26. Transgenic cardiomyocytes as claimed in claim 25, containing at least one vector as claimed in claim 17.

27. Transgenic cardiomyocytes which can be obtained using a process as claimed in one of claims 19 to 23.

28. Transgenic cardiomyocytes which can be obtained using a process as claimed in claim 18 or 20 to 23.

29. Use of transgenic cells as claimed in one of claims 24 to 28, for preferably autologous cell transplantation, or for gene therapy, as especially for cell-mediated gene transplantation.

30. Use of an expression cassette as claimed in one of claims 1 to 15 or of a vector as claimed in claim 16 or 17 for genetic alteration of pluripotent precursor cells of a mammal.

31. Use of an expression cassette as claimed in one of claims 1 to 15 or of a vector as claimed in claim 16 or 17 for producing in vitro differentiated somatic cells of a mammal.
